(19) United States
(12) Patent Application Publication
(10) Pub. No.: US 2018/0187150 A1
(43) Pub. Date: Jul. 5, 2018

(54) Method for the Cryopreservation of Tumour-Infiltrating Lymphocytes

(71) Applicant: Chu Nantes, Nantes (FR)

(72) Inventor: Joffrey De Larichaudy, Malakoff (FR)

(73) Assignee: Chu Nantes, Nantes (FR)

(21) Appl. No.: 15/740,630

(22) PCT Filed: Jun. 30, 2016
(86) PCT No.: PCT/FR2016/051631

§ 371 Date: Dec. 28, 2017

(30) Foreign Application Priority Data
Jun. 30, 2015 (FR) 15 56164

(51) Int. Cl. C12N 5/0783; A01N 1/02

(52) U.S. Cl. CPC C12N 5/0636; A01N 1/0221

(57) ABSTRACT

The invention relates to a method for the cryopreservation of at least one sample of tumour-infiltrating lymphocytes, comprising the following steps: i) the in vitro culture of tumour-infiltrating lymphocytes using a collection of samples of in-transit skin nodules, ganglion or metastasis, from a patient with a stage III or IV melanoma, said step comprising the emergence of the tumour-infiltrating lymphocytes contained in the sample of in-transit skin nodules, ganglion or metastasis, followed by the stimulation of the tumour-infiltrating lymphocytes resulting from the emergence step, and, subsequently, the amplification of the stimulated tumour infiltrating lymphocytes; ii) the mixing of at least one sample obtained in step (i) with a composition comprising, in a physiologically acceptable medium, a) human albumin serum, b) at least one saccharide, and c) at least two ingredients selected from among DMSO, L-cysteine, coenzyme Q10 and C3-C5 alkanediols; and subsequently iii) the freezing of the mixture obtained in step (ii).

The present invention relates to a process for the cryopreservation of at least one sample of tumor-infiltrating lymphocytes, comprising the following steps:
i) in vitro culture of tumor-infiltrating lymphocytes from a sample of in-transit cutaneous nodules, lymph-node or metastasis from a patient suffering from a stage 3 or 4 melanoma, comprising specific steps,
ii) mixing at least one sample obtained in step i) with a composition comprising, in a physiologically acceptable medium:
   a) human serum albumin,
   b) at least one saccharide, and
   c) at least two ingredients selected from DMSO, L-cysteine, coenzyme Q10 and C3-C5 alkanediols, then
iii) freezing the mixture obtained in step ii).

Cryopreservation is a process in which biological samples are stored at very low temperature. The cryopreservation of biological material is generally carried out by freezing said material, in an appropriate support, such as a tube or a vial made of glass or plastic material (generally referred to as “straws” or cryotubes in the field of cryopreservation), said support being suitable for long-term storage at low temperature.

However, cryopreservation poses a certain number of problems and technical constraints. Cell damage may especially arise during the thawing, leading to apoptosis or the bursting of the cells. In addition, the survival of cryopreserved cells may depend on the conditions and techniques used during the freezing. Controlling the rate of cooling is important: cooling at a low rate enables ordered crystallization of the freezable water outside the cells—the cells become dehydrated, shrink, and the water leaves the cell. In the opposite scenario, the formation of intracellular ice leads to the destruction of membrane structures, which is lethal for the cell.
For the majority of mammalian cells, as is the case for the products and medications for cell therapy, whether the cells are genetically modified or not, it is also vital to use cryo-protectants in order to preserve cell integrity and functionality.

Currently, manufacture on an industrial scale (European or worldwide, especially) of products for cell therapy poses new problems, such as the stability of the finished product administered and variability linked to the starting biological material.

In the majority of cases, the finished product is packaged:
fresh in a liquid medium (of albumin or saline solution type) with preservation limited to a few hours or days, or else
frozen in a simple formulation based on DMSO, which is not very stable and not very effective in the long term. The not inconsiderable amount of dead cells, of debris with potentially immunogenic effects, and the toxicity for the patient of the excipients commonly used are all limiting factors. Washing the cells to remove these toxic elements (whether biological or not) is commonly advised: these solutions are not compatible with distribution on an industrial scale. Moreover, they do not afford much flexibility in terms of administration and storage, unlike other “conventional” classes of medication.

There is therefore a need to develop a product and/or medication for cell therapy which is stable in the long term (i.e. for several months), which is easy to use, which can be directly injected, and which is non-toxic.

The present invention makes it possible to meet this need. Indeed, the cryopreservation process according to the invention makes it possible to obtain products for cell therapy comprising tumor-infiltrating lymphocytes, which are ready to use (i.e. ready to be injected without washing, which avoids all additional handling operations causing decreased viability and loss of cells), stable in the long term, easy to use, and non-toxic.

The present invention therefore relates to a process for the cryopreservation of at least one sample of tumor-infiltrating lymphocytes, comprising the following steps:
i) in vitro culture of tumor-infiltrating lymphocytes from a sample of in-transit cutaneous nodules, lymph-node or metastasis from a patient suffering from a stage 3 or 4 melanoma, comprising the emergence of said tumor-infiltrating lymphocytes contained in the sample of in-transit cutaneous nodules, lymph-node or metastasis, then the stimulation of the tumor-infiltrating lymphocytes resulting from the emergence step, then finally the amplification of the stimulated tumor-infiltrating lymphocytes, in order to obtain at least one sample of tumor-infiltrating lymphocytes,
ii) mixing at least one sample obtained in step i) with a composition comprising, in a physiologically acceptable medium:
   a) human serum albumin,
   b) at least one saccharide, and
   c) at least two ingredients selected from DMSO, L-cysteine, coenzyme Q10 and C3-C5 alkanediols, then
iii) freezing the mixture obtained in step ii).

The present invention also relates to the use of a composition comprising, in a physiologically acceptable medium:
a) human serum albumin,
b) at least one saccharide, and
c) at least two ingredients selected from DMSO, L-cysteine, coenzyme Q10 and C3-C5 alkanediols, for the cryopreservation of at least one sample of tumor-infiltrating lymphocytes (also referred to as TILs),
said sample being obtained by in vitro culture of tumor-infiltrating lymphocytes from a sample of in-transit cutaneous nodules, lymph-node or metastasis from a patient suffering from a stage 3 or 4 melanoma, said culture comprising the emergence of said tumor-infiltrating lymphocytes contained in the sample of in-transit cutaneous nodules, lymph-node or metastasis, then the stimulation of the tumor-infiltrating lymphocytes resulting from the emergence step, then finally the amplification of the stimulated tumor-infiltrating lymphocytes.

Tumor-infiltrating lymphocytes (TILs) is intended to mean tumor-infiltrating lymphocytes, whether genetically modified or not.

The composition used in the cryopreservation process according to the invention (step ii)) therefore comprises, in a physiologically acceptable medium:
a) human serum albumin,
b) at least one saccharide, and
c) at least two ingredients selected from DMSO, L-cysteine, coenzyme Q10 and C3-C5 alkanediols.

“Physiologically acceptable medium” is intended to mean an aqueous medium comprising electrolytes. Electrolytes are, for example, salts of sodium, potassium, magnesium, and/or calcium, with anions of chloride, carbonate, hydroxide or caprylate type. The pharmaceutically acceptable medium is preferably an aqueous medium comprising sodium chloride and sodium caprylate.

The composition used in the cryopreservation process according to the invention comprises human serum albumin (compound a)). This protein is a high molecular weight (approximately 65 kDa) plasma protein. It is the most abundant protein in the plasma; the normal mean concentration thereof is from 38 to 48 g/l. It makes it possible to buffer the pH and to maintain osmolarity. The purity thereof is preferably 95%. It is also possible to use one or more fragments and/or derivatives of human serum albumin, said fragments or derivatives being non-immunogenic and having an oncotic property similar to human serum albumin. Preferably, the human serum albumin, the fragments and/or derivatives thereof, is present in an amount of between 2 and 10% by weight relative to the total weight of composition, preferably between 2.5 and 6% by weight, preferably between 3.5 and 4.5% by weight. In the present application, unless indicated otherwise, the amounts are mentioned by weight relative to the total weight of composition.

The composition used in the cryopreservation process according to the invention also comprises at least one saccharide (compound b)). The saccharide improves cell survival and function by preserving osmotic equilibrium. A fraction penetrates the cells and makes it possible to stabilize the membrane structures. The saccharide is preferably selected from monosaccharides, disaccharides and trisaccharides.

The monosaccharides are preferably selected from glucose, galactose, fructose and mannose. The disaccharide preferably has the formula A-B, wherein A and B are each independently selected from glucose, fructose and mannose. The saccharide is preferably a disaccharide. The disaccharide is preferably a glucose dimer. More preferentially, the disaccharide is selected from trehalose and sucrose. More preferentially, the disaccharide is trehalose.

The trisaccharides are preferably selected from raffinose (trimer of galactose, glucose and fructose), maltotriose and isomaltotriose (glucose trimers).

The saccharide is preferably present in the composition according to the invention at a concentration of between 0.05 M and 1 M, preferably between 0.07 M and 0.5 M, preferably between 0.08 M and 0.12 M.

The composition used in the cryopreservation process according to the invention finally comprises at least two ingredients selected from DMSO, L-cysteine, coenzyme Q10 and C3-C5 alkanediols (compounds c)).

DMSO, or dimethylsulfoxide, is a polar aprotic organic solvent of formula CH₃—SO—CH₃. It is an intracellular cryo-protectant, the main aim of which is to replace the intracellular liquid, and which thus makes it possible to prevent the formation of ice crystals and the osmotic stress inherent to the phases of freezing/thawing which cause membrane structures to burst. It is preferably present in the composition according to the invention in an amount of between 2 and 15% by weight, preferably between 2.5 and 4.5% by weight.

L-cysteine is an amino acid having a thiol group -SH. It is preferably present in the composition at a concentration of between 0.05 mM and 5 mM, preferably between 0.5 mM and 2 mM.

Coenzyme Q10, also referred to as ubiquinone, is a chemical compound comprising a quinone group. Its chemical name is 2,3-dimethoxy-5-methyl-6-decaprenylbenzoquinone. It is preferably present in the composition in an amount of between 0.005 and 1% by weight, preferably between 0.007 and 0.5% by weight, preferably between 0.007 and 0.1% by weight. The C3-C5 alkanediol is preferably a linear, branched or cyclic alkane comprising from 3 to 5 carbon atoms, and 2 hydroxyl groups. It is preferably selected from linear alkanes comprising from 3 to 5 carbon atoms, and 2 hydroxyl groups. More preferentially, it is selected from propane-1,2-diol, pentane-1,5-diol and butane-2,3-diol. The C3-C5 alkanediol is preferably propane-1,2-diol (also referred to as propylene glycol).

The C3-C5 alkanediol is preferably present in the composition in an amount of between 3 and 10% by volume, preferably between 3 and 7% by volume.

The composition used in the cryopreservation process according to the invention preferably comprises at least the two following ingredients as compounds c):
DMSO and L-cysteine, or
coenzyme Q10 and a C3-C5 alkanediol, preferably propylene glycol.

The composition used in step ii) of the cryopreservation process according to the invention preferably comprises, in a physiologically acceptable medium:
a) human serum albumin, preferably in an amount of between 2.5 and 6% by weight,
b) a saccharide, preferably trehalose, preferably at a concentration of between 0.07 M and 0.5 M, and
c) DMSO and L-cysteine, preferably respectively in an amount of between 2 and 5% by weight and at a concentration of between 0.5 mM and 2 mM.

Alternatively, the composition used in step ii) of the cryopreservation process according to the invention preferably comprises, in a physiologically acceptable medium:
a) human serum albumin, preferably in an amount of between 2.5 and 6% by weight,
b) a saccharide, preferably trehalose, preferably at a concentration of between 0.07 M and 0.5 M, and
c) coenzyme Q10 and propane-1,2-diol, preferably respectively in an amount of between 0.007 and 0.1% by weight and in an amount of between 3 and 10% by volume.

The cryopreservation process according to the invention is particularly beneficial, and aims to cryopreserve at least one sample of tumor-infiltrating lymphocytes. Indeed, as demonstrated in the example, the composition used in step ii) of the cryopreservation process according to the invention makes it possible to durably and effectively cryopreserve TILs (especially up to 4 months after thawing).

Tumor-infiltrating lymphocytes (or TILs) are lymphocytes typically found in in-transit cutaneous nodules, lymph-node or metastasis from patients suffering from advanced-stage melanoma (stage 3 or 4). They are involved in the death of tumor cells, and are currently being tested in various clinical trials as products for cell therapy. TILs generally originate from patients themselves; they are autologous. For this, they are taken from the patient, isolated from the tumor and are multiplied in vitro.

More specifically, the sample of TILs used in the process according to the present invention is obtained by a step i) of in vitro culture comprising the following steps:
emergence of tumor-infiltrating lymphocytes contained in a sample of in-transit cutaneous nodules, lymph-node or metastasis. This emergence is also referred to as “primary culture” or “exit”; it is the step during which the tumor-infiltrating lymphocytes, initially contained in the sample of in-transit cutaneous nodules, lymph-node or metastasis, move into the culture medium and are cultured in vitro,
stimulation of the tumor-infiltrating lymphocytes resulting from the emergence step, then finally
amplification of the tumor-infiltrating lymphocytes resulting from the stimulation step.

The step of emergence of the TILs is preferably carried out by primary culture of the sample of in-transit cutaneous nodules, lymph-node or metastasis from a patient suffering from a stage 3 or 4 melanoma, preferably in a closed culture container. Indeed, the source of the TILs is a sample of in-transit cutaneous nodules, lymph-node or metastasis from the patient suffering from stage 3 or 4 melanoma: it may be sampled by biopsy (sample taken from a tumor). The primary culture is preferably carried out in a serum-free selective culture medium of X-VIVO 15® type (sold by Cambrex Corp) supplemented with interleukin-2 (IL-2). “Closed culture container” is intended to mean a system which makes it possible to maintain cells in culture in a suitable medium, without said cells being directly in contact with the external environment. Thus, the container is closed, i.e. its interior volume is not in contact with the ambient air, especially during the culture, when the TILs and the culture medium are introduced into the container.

After emergence of the TILs, the latter undergo a step of stimulation, preferably in a compartmentalized closed culture container. This enables a significant quantitative increase in the global yield of TILs (up to 2.5 times) compared to the same technique carried out in an open or non-compartmentalized system. It also enables a qualitative increase in the TILs produced. Indeed, among the TILs produced, the proportion of CD8+ TILs is greater than that produced (85% versus 60%) under conventional conditions, and has a greater specific cytotoxic activity with regard to the autologous tumor line (14% versus 2%).

According to a preferred embodiment, the TILs are stimulated in the presence at least of non-proliferating irradiated allogeneic feeder cells. These allogeneic feeder cells are made non-proliferating, for example, by irradiation. The TILs are preferably stimulated by coculture with a mixture formed of (non-proliferating) mononuclear cells originating from healthy human donor blood and of (non-proliferating) B cells transformed by the Epstein-Barr virus, preferably the line LAZ388.

The non-proliferating allogeneic feeder cells and the TILs are preferably co-cultured in a serum-free selective culture medium of X-VIVO 15® type (sold by Cambrex Corp), supplemented with interleukin-2 (IL-2). In addition, during the stimulation step, a mitotic agent such as PHA-L (type L phytohemagglutinin), which promotes the amplification of the lymphocytes, is preferably introduced into the closed stimulation container in parallel to the lymphocytes.

Generally, the compartmentalized closed culture container comprises at least two compartments, one referred to as the technical chamber, the other referred to as the pacification chamber, which are in communication with one another, and at least one port opening into one of the compartments. The TILs and the non-proliferating irradiated allogeneic feeder cells are introduced suspended in a culture medium via said port, and the culture medium is renewed via the, or another, port.

The step of stimulating the TILs is followed by an amplification step. Typically, the stimulated TILs are harvested then transferred into a culture system. The bags are incubated, and new medium supplemented with IL-2 is added to each bag twice a week. Bags are thus obtained, the concentration of TILs of which is between 1.10⁶ and 3.10⁶ lymphocytes/ml.

It is possible to carry out the steps of emergence, stimulation and amplification in a different closed culture container from one step to another. In this case, the transfer from one closed container to another is carried out under sterile conditions, for example by connecting, by means of a sterile connection apparatus of SCD® type by Terumo, the tubing serving as ports. As a variant, the closed containers for emergence, stimulation and amplification are pre-connected to one another at the moment of manufacture, so as to form a closed system. Such a system makes it possible to carry out the complete culture of the T lymphocytes in a closed system, thereby limiting the risk of contamination.

In the cryopreservation process according to the invention, the step ii) of mixing the sample of TILs with the composition described above is typically carried out by dilution. The sample of TILs is preferably taken up in the compound a) in liquid form, preferably for 50% of the volume, then the compounds b) and c), preferably mixed together beforehand to obtain a 2× concentration solution, are added. The mixing of step i) may be carried out at around 4° C., or at room temperature (i.e. 20° C.).

The sample of TILs preferably undergoes centrifugation, the supernatant is withdrawn, and the pellet is suspended in the composition described above.

With regards to the freezing (step iii)) of the cryopreservation process according to the invention, it is preferably carried out over a drop in temperature from +4° C. or room temperature down to a temperature of between −100° C. and −160° C. The step of freezing (step ii)) is preferably carried out down to a temperature of between −100° C. and −180° C., preferably of between −140° C. and −160° C.

The sample is then stored at a temperature of generally less than −130° C.

Preferably, the freezing iii) is carried out by placing the mixture obtained in step ii) in a container submerged in a mixture of isopropyl alcohol at +4° C., everything being brought to a temperature of between −70° C. and —100° C. This system (“freezing in a Nalgene box”) enables, by virtue of the slow cooling of the alcohol, a virtually linear drop in temperature of between −1° C. and −2° C. per minute. Alternatively, preferably, the freezing iii) is carried out by means of a programmed freezer. Such freezers are especially sold by Air Liquide or Cryobiosystem. They especially enable a more reproducible drop in temperature and more complex schemes.

Preferably, the freezing iii) is carried out, especially by means of a programmed freezer, according to the following steps:
placing the mixture obtained in step ii) at a temperature of +4° C.; then
decreasing the temperature by 1° C. per minute from +4° C. to −40° C.; then
decreasing the temperature by 10° C. per minute, from −40° C. to −150° C., to reach a final storage temperature of approximately −150° C. on average, in a container of liquid or gaseous nitrogen.

The frozen product thus obtained may be kept for several months at approximately −150° C.

The invention will now be exemplified by means of the following nonlimiting examples.

### EXAMPLE 1

### Preparation and Cryopreservation of a Sample of TILs According to the Process of the Invention

The TILs product constitutes an Advanced Therapy Medicinal Product (ATMP) of autologous nature. It consists of living cells intended exclusively for the patient who is the donor of the starting material (in this case the lymph node taken from the patient). The TILs from a patient, after ex vivo amplification, will be reinjected into this same patient: this is an autologous system.

The process is as follows:

The starting raw material consists of tumor-infiltrating lymphocytes infiltrating metastatic tumors isolated from the lymph nodes of patients suffering from cutaneous malignant melanoma.

The patients undergo a virological screening beforehand (CMV, hepatitis B and C, HIV, HTLV, EBV and parvovirus B19).

### Step i) of the Process: Obtaining a Sample of TILs:

The piece(s) of the samples taken (cutaneous, pulmonary and/or from lymph nodes), invaded by metastatic-stage melanoma cells, are collected aseptically per-operatively then placed “dry” (without conditioning in a transport medium) in sterile containers.

They are then cut into fragments of 1 to 2 mm. The fragments of each tumor sample taken are then frozen in cryotubes, to await their preparation.

### Step of Emergence of the TILs:

The cryotubes of tumor fragments are thawed and placed in wells of 12-well culture plates containing X VIVO 15® culture medium and IL-2. The plates are incubated at 37° C. and 5% CO₂.

The wells are examined under inverted microscope and the culture medium with IL-2 added to it is renewed by half the volume, twice a week.

After 10-12 days of culture, all the TILs exiting the tumor pieces are harvested, and a defined amount of these TILs is used for stimulation.

### Step of Stimulation of the TILs:

The viable TILs obtained beforehand are placed in coculture, in the presence of PHA-L, with irradiated feeder cells composed of allogeneic lymphocytes (originating from a secure bank of mononuclear cells constituted from ring residues from a CPA donation kit from 3 healthy donors) and of irradiated LAZ cells (allogeneic B line transformed by the Epstein-Barr virus: line LAZ388).

The irradiation of the feeder cells is necessary to block the proliferation of these cells without detrimentally affecting their feeder effects (production of soluble factors, expression of markers on their surface, etc.), which are important in the activation and proliferation of the TILs.

TILs (1.8.10⁶ cells) and feeder cells (allogeneic lymphocytes: 240.10⁶ cells; LAZ388: 120.10⁶ cells) are cultured in 60 96-well plates in an X VIVO 15® culture medium and IL-2. The plates are incubated at 37° C. and 5% CO₂.

Twice a week, the upper third of the medium present in the wells is removed and replaced with new medium and IL-2. After 10 to 13 days, the TILs have proliferated and form an opaque pellet at the bottom of the wells. The content of all the wells is then collected.

### Step of Amplification of the TILs:

The TILs harvested from the 96-well plates are transferred into culture bags and the cell concentration is readjusted. The bags are incubated, and new X VIVO 15® medium and IL-2 are added twice a week to each bag so as to adjust the concentration of the TILs in the bags to 1.10⁶ lymphocytes/ml.

Step ii) of the Process: Mixing the Sample of TILs with the Cryopreservation Composition:

After 10 days of bag culture (amplification step), the volume of cell suspension harvested from the bags of TILs is reduced by centrifugation to concentrate the TILs, which are washed in buffered physiological saline (0.9% NaCl). The pellet containing the cells is then mixed with one of the following compositions:
“composition A”, comprising 3.5% DMSO, 1 mM L-cysteine, 0.1 M trehalose and 4% human serum albumin (HSA); or
“composition B”, comprising 5% propylene glycol, 0.01% coenzyme Q10, 0.1 M trehalose and 4% human serum albumin (HSA).
   Step iii) of the Process: Freezing the Mixture Obtained:

Each mixture obtained at the end of step ii) is frozen at −150° C.

Each product is stored in frozen form in container of gaseous nitrogen at a temperature below −150° C., to await thawing and administration to the patient.

Each cryotube produced contains:
1. 75×10⁶ viable cells
2. A constant cell concentration fixed at 75×10⁶ viable cells/ml.
3. The volume of the product is 1 ml.
4. The cellular composition thereof is as follows:
   a. Active substance:
      i. More than 99% CD45⁺ cells.
      ii. More than 50% CD45⁺ CD3⁺ CD8⁺ immunocompetent T lymphocytes co-expressing IFNg and CD107a in response to CD3⁺ CD25⁺ activation (=potentiality test).
   b. Impurities:
      i. Traces of HMB45⁺ tumor cells (<1% limit of detection of the analytical method used).
      ii. Traces of CD45⁺ CD3⁻ CD19⁺ B lymphoid cells (<1% limit of detection of the analytical method used).
      iii. Traces of CD45⁺ CD3⁻ CD56⁺ and/or CD16⁺ NK cells (<1% limit of detection of the analytical method used).
      iv. Cell debris (=detection by flow cytometry or by residual DNA assay).

### EXAMPLE 2

### Effectiveness of the Compositions in the Cryopreservation of TILs According to the Invention

The cells used here are TILs obtained as described in step i) of example 1.

### The Formulation:

The compositions according to the invention are tested in comparison to:
a simple formulation (negative control), which is known and widely used in hematopoietic cell transplantation units, but which has a limited storage capacity: this is a mixture of 10% DMSO+4% human serum albumin (HSA); and
a fresh formulation, in which the cells are placed in 4% human serum albumin and analyzed directly, which is the positive control.

2 compositions according to the invention are evaluated:
“compositionA”, comprising 3.5% DMSO, 1 mM L-cysteine, 0.1 M trehalose and 4% human serum albumin (HSA); and
“composition B”, comprising 10% propylene glycol, 0.01% coenzyme Q10, 0.1 M trehalose and 4% human serum albumin (HSA).

### Method of Freezing:

freezing in a Nalgene box is the known manual reference: the tubes are placed in a hermetic box filled with a mixture of isopropane-alcohol at +4° C., then everything is placed at −80° C. and the slow cooling of the alcohol enables a virtually linear drop in temperature of between −1° C. and −2° C. per minute;
automated freezing by means of the CRF (programmed freezer) is controlled electronically; it enables linear or non-linear curves to be produced, in order to optimize the freezing yields. This method is reproducible.

The cells are evaluated at the final point after thawing following 1 month of storage in frozen form at −150° C. maximum.

### Materials and Methods

Cryoprotectants and Reagents

| Material | Manufacturer |
|---|---|
| Propylene glycol | NA |
| Trehalose dihydrate | NA |
| Human albumin | LFB |
| DMSO | Wak-chemie or Miltenyi |
| Coenzyme Q10 | NA |
| CFSE (carboxyfluorescein diacetate | Life technologies |
| succinimidyl ester, fluorescent | |
| intracellular stain) | |
| Antibodies used | |
| CD3/CD28 beads | |

### Apparatus

| Material | Supplier |
|---|---|
| CRF (programmed freezer) | Cryobiosystem (range- digitcool) |
| | Air Liquide (freezal range) |
| Flow cytometer | NA |
| Cell counter | NucleoCounter NC200 (Chemometec) |
| | type |

### Methods

Starting from a single starting material, which is a fresh cell suspension resulting from a continuous cell culture/from a normal process, 4 study arms are envisaged:
Common T0 arm =This relates to fresh T0 cells analyzed before centrifugation in culture medium.
Reference arm (negative control) (tubes 1)=this relates to cells frozen in 10% DMSO+4% HSA, according to a staged method, by means of a Nalgene box (manual method). This method is described as being moderately stressing for the cells during a prior study, in comparison to non-frozen positive control cells.
Positive control arm T1=this relates to non-frozen cells kept in 4% human serum albumin to simulate a fresh, injectable formulation.
Test arm (tubes 2)=this relates to cells frozen in a composition according to the invention (composition A or B) by the method by CRF freezing (tubes 3).

More specifically, the protocol is as follows:
The fresh cells of the T0 are under suitable culture conditions defined by Nantes UTCG (unit for cell and gene therapy). Enumerate the TIL cells, measure their viability and their proliferation (700.10⁶ living cells are necessary for all the conditions): this establishes the T0=non-formulated fresh cells reference
This experiment was carried out 3 times independently (on 3 different finished products originating from 3 different donors)
Produce 3 cryotubes of 75.10⁶ cells for each condition
The T0 and T1 are produced a single time (10⁷ cells per condition)
Distribute the suspension in one 250 ml flask, i.e. 225.10⁶ living cells per tube
Centrifuge at 400 g for 8 minutes
Each cryotube has a final suspension volume of 1 ml and a cell concentration of 75×10⁶ living cells/ml
After centrifugation, carefully remove the supernatant by means of a micropipette or a suction pump
Take the cells up in 1.5 ml of 4% HSA for the conditions for tubes 1 and 2
Transfer the cell suspension (=500 μl) into the 3 numbered cryotubes
Add 500 μl of freezing medium dropwise at a concentration of 2×
Close the cryotubes
The tubes 2 are placed in a composition according to the invention then are incubated for 10 minutes at +4° C. for the formulation based on DMSO and 30 minutes at +4° C. for the formulation based on C3-C5 alkanediols, then frozen in the CRF.
The tubes 1 are frozen in a Nalgene box, stabilized beforehand at +4° C.

The cryotubes of the test arm are frozen by means of a Controlled Rate Freezer (CRF), in which they undergo a programmed drop in temperature, after a prior incubation at +4° C., of −1° C. per minute down to a stage of −40° C., then a rapid freezing of −10° C./minute down to the storage temperature (−130° C.). The storage is then carried out in a container of gaseous nitrogen at −150° C.

The cryotubes of the reference arm are placed in a Nalgene box (=freezing box), placed at +4° C. Everything is transferred directly to the freezer at −80° C. overnight. The cryotubes are transferred to a container of gaseous nitrogen for storage after at most 48 hours.

### Thawing:

After at least 1 month, the cryotubes are thawed by a rapid method. To this end, the cryotube is removed from the nitrogen container then transported to the Quality Control centers using a Nalgene box at −80° C. Upon arrival, it is immediately submerged in a water bath at +37° C. until all ice disappears (after approximately 1 minute). Everything is directly numbered and analyzed according to the tests described in the “evaluation criteria” paragraph below. The same analysis is carried out 4 hours later for the positive control T1, formulated in human serum albumin.

Naming the Cryotubes:

| | | Medium 1 | | | |
|---|---|---|---|---|---|
| | | (negative | | | |
| | | control - | | | |
| | | 10% DMSO | Medium 2 | | |
| | Fresh | Nalgene | (positive | Medium 3 | Medium 4 |
| Medium | T0 cells | freezing) | control T1) | (DMSO) | (PG) |
| No. of | NA | 1-A to C | NA | 2-A to C | 2′-A to C |
| vials | | | | | |

NA: not applicable
Cell concentration: The cell concentration is identical for all the vials, i.e. 75 · 10⁶ cells in one milliliter, per vial.

### Evaluation Criteria

The evaluation criteria are:
Viability by automatic method upon thawing and after 4 hours (nucleocounter type).
Numbering of the living cells upon thawing and after 4 hours.
Test of proliferation of the cells over 3 days (maintained over 2 days after re-culturing: by CFSE test).

The cells are analyzed after rapid thawing in a water bath temperature-controlled to +37° C. The thawing is rapid, until all the ice has disappeared (which simulates the preparation of a product suitable for human injection), then the tube is kept at room temperature (15° C.-25° C.) for 4 hours in order to verify the thawed stability.

### Results

The results are given in tables 1 to 3 below.

In order for a composition to be retained for the TILs, it must give a result greater than 85% of the positive control (T1: cells formulated in 4% serum albumin, HSA) and significantly greater than the negative control (10% DMSO +4% HSA) upon thawing, and the decrease must not exceed 20% of the positive control after 4 hours.

In the case of the CD8/CD4 ratio, which documents the stability of the cell populations in the finished product, the ratio must not vary by more than 40%.

TABLE 1
post-thawing viability in 4% HSA determined by manual counting or eosin

| | | | T1 | | | |
|---|---|---|---|---|---|---|
| | | T0 | (positive | Composition | Composition | Negative |
| | | (reference) | control) | A | B | control |
| Batch 1 | Viability in % | 86.80 | 79.90 | 81.60 | 85.00 | 72.10 |
| | Recovery rate T0 | 100.00 | 92.05 | 94.01 | 97.93 | 83.06 |
| | Viability in % of T0 | 0.0 | −7.9 | −6.0 | −2.1 | −16.9 |
| Batch 2 | Viability in % | 85.90 | 74.11 | 73.40 | 77.30 | 56.30 |
| | Recovery rate T0 | 100.00 | 86.27 | 85.45 | 89.99 | 65.54 |
| | Viability in % of T0 | 0.0 | −13.7 | −14.6 | −10.0 | −34.5 |
| Batch 3 | Viability in % | 85.88 | 82.90 | 75.50 | 80.70 | 67.80 |
| | Recovery rate T0 | 100.00 | 96.53 | 87.91 | 93.97 | 78.95 |
| | Viability in % of T0 | 0.0 | −3.5 | −12.1 | −6.0 | −21.1 |
| means | Viability in % | 86.19 | 78.97 | 76.83 | 81.00 | 65.40 |
| | Viability in % of T0 | 0.0 | −8.4 | −10.9 | −6.0 | −24.1 |
| | Recovery rate T0 | 100.0 | 91.6 | 89.1 | 94.0 | 75.9 |
| | Standard deviation | 0.0 | 5.1 | 4.4 | 4.0 | 9.2 |
| | recovery rate | | | | | |
| | Recovery rate T1 | 109.1 | 100.0 | 97.3 | 102.6 | 82.8 |
| | Standard deviation | 0.5 | 4.5 | 4.3 | 3.9 | 8.2 |
| | (mean) | | | | | |
| | SEM | 0.3 | 2.6 | 2.5 | 2.2 | 4.7 |

After 4 h at room temperature (RT), the mean viability obtained with the composition A is 69.20% (87.6% of T1) and with composition B it is 70.97% (89.9% of T1), which is significantly greater than the viability of the negative control (53.00%).

TABLE 2
proliferation of TILs over 2 days following CFSE staining

| | | | | | | Negative |
|---|---|---|---|---|---|---|
| | | T0 | T1 | Composition A | Composition B | control |
| Batch 1 | proliferating cells in % | 64.80% | 60.12% | 75.49% | 83.70% | 66.19% |
| | Recovery rate T0 | 100.00% | 92.78% | 116.50% | 129.17% | 102.15% |
| | in % of T0 | 0.0 | −38.2% | −22.4% | −14.0% | −32.0% |
| Batch 2 | proliferating cells in % | 96.54% | 96.48% | 93.53% | 90.89% | 94.02% |
| | Recovery rate T0 | 100.00% | 99.94% | 96.88% | 94.15% | 97.39% |
| | in % of T0 | 0.0 | −0.9% | −3.9% | −6.6% | −3.4% |
| Batch 3 | proliferating cells in % | 98.34% | 97.92% | 92.53% | 91.76% | 59.00% |
| | Recovery rate T0 | 100.00% | 99.57% | 94.09% | 93.31% | 60.00% |
| | in % of T0 | 0.0% | 11.0% | 4.9% | 4.0% | −33.1% |
| Means | proliferating cells in % | 81.57% | 79.02% | 87.18% | 88.78% | 73.07% |
| | in % of T0 | 0.0% | −3.13 | 6.88 | 8.84 | −10.42 |
| | in % of T1 | 3.23 | 0.00 | 10.33 | 12.36 | −7.53 |
| | Standard deviation | 18.87% | 21.42% | 10.14% | 4.42% | 18.50% |
| | SEM | 10.89% | 12.37% | 5.85% | 2.55% | 10.68% |
| | Recovery rate T1 | | 100.00% | 110.33% | 112.36% | 92.47% |
| | Recovery rate T0 | 100.0% | 96.9% | 106.9% | 108.8% | 89.6% |
| | Recovery rate standard | 0.0% | 4.0% | 12.2% | 20.5% | 23.1% |
| | deviation | | | | | |

TABLES 3
characterizaion of the cells by flow cytometry

| | | | Composition | Composition | Negative |
|---|---|---|---|---|---|
| | T0 | T1 | A | B | control |
| Batch 1 | | | | | |
| CD45+ | | | | | |
| CD45+ CD3+ CD4+ | 66.68% | 67.18% | 60.90% | 61.00% | 60.70% |
| CD45+ CD3+ CD8+ | 28.72% | 28.33% | 30.50% | 30.90% | 31.20% |
| Control | | | OK | OK | OK |
| CD45+ CD3+ CD25+ | 14.49% | 12.55% | 14.90% | 15.30% | 14.50% |
| CD8/CD4 ratio | 0.43 | 0.42 | 0.50 | 0.51 | 0.51 |
| Batch 2 | | | | | |
| CD45+ | 99.86% | 99.44% | 99.92% | 99.60% | 99.09% |
| CD45+ CD3+ CD4+ | 47.30% | 46.65% | 56.30% | 54.33% | 55.89% |
| CD45+ CD3+ CD8+ | 48.26% | 47.63% | 20.12% | 20.07% | 21.33% |
| Control | 0.56% | 0.39% | 0.74% | 0.52% | 0.57% |
| CD45+ CD3+ CD25+ | 16.47% | 12.91% | 50.82% | 47.69% | 38.48% |
| CD8/CD4 ratio | 1.02 | 1.02 | 0.36 | 0.37 | 0.38 |
| Batch 3 | | | | | |
| CD45+ | 99.88% | 99.88% | 99.87% | 99.85% | 99.74% |
| CD45+ CD3+ CD4+ | 77.14% | 77.46% | 76.11% | 76.70% | 70.24% |
| CD45+ CD3+ CD8+ | 16.70% | 16.98% | 17.09% | 16.58% | 19.25% |
| Control | 0.38% | 0.38% | 0.54% | 0.50% | 0.46% |
| CD45+ CD3+ CD25+ | 35.95% | 30.81% | 43.78% | 43.16% | 40.11% |
| CD8/CD4 ratio | 0.22 | 0.22 | 0.22 | 0.22 | 0.27 |
| Means | | | | | |
| CD45+ CD3+ CD4+ | 63.71% | 63.76% | 64.44% | 64.01% | 62.28% |
| CD45+ CD3+ CD8+ | 31.23% | 30.98% | 22.57% | 22.52% | 23.93% |
| CD45+ CD3+ CD25+ | 22.30% | 18.76% | 36.50% | 35.38% | 31.03% |
| CD25+ | 11.86% | 10.44% | 19.03% | 17.54% | 14.34% |
| Recovery rate T1 | 118.9% | 100% | 194.6% | 188.6% | 165.4% |
| Recovery rate | 0.0% | 4.5% | 113.7% | 102.3% | 74.0% |
| standard deviation | | | | | |
| CD8/CD4 ratio | 0.56 | 0.55 | 0.36 | 0.36 | 0.39 |

| | | | | | | Negative |
|---|---|---|---|---|---|---|
| | | T0 | T1 | Composition A | Composition B | control |
| Batch 1 | % of IFNg⁺/CD107⁺ | 66.6 | 71.96 | 73.0 | 66.5 | 64.0 |
| | cells among CD8⁺ | | | | | |
| | Recovery rate T1 | 92.6 | 100.0 | 101.4 | 92.5 | 88.9 |
| Batch 2 | % of IFNg⁺/CD107⁺ | 77.0 | 83.68 | 63.1 | 64.7 | 53.1 |
| | cells among CD8⁺ | | | | | |
| | Recovery rate T1 | 92.0 | 100.0 | 75.4 | 77.3 | 63.4 |
| Batch 3 | % of IFNg⁺/CD107⁺ | 85.3 | 85.9 | 83.7 | 78.8 | 62.7 |
| | cells among CD8⁺ | | | | | |
| | Recovery rate T1 | 99.3 | 100.0 | 97.4 | 91.8 | 73.0 |
| Means | % of IFNg⁺/CD107⁺ | 76.3 | 80.5 | 73.2 | 79.9 | 59.9 |
| | cells among CD8⁺ | | | | | |
| | Recovery rate (T1) | 94.8% | 100.0% | 91.0% | 87.0% | 74.4% |
| | Standard deviation | 4.1 | 0.0 | 14.0 | 8.6 | 12.9 |

In conclusion, the 2 formulations tested achieve the specifications set (less than 15% difference relative to the positive control: fresh cells formulated in 4% serum albumin, which is an injectable solution).

For the formulation A:
The viability achieves 97.3% of the value of the positive control upon thawing and is maintained at 87.6% after 4 hours (76.8% upon thawing, then 69.2% after 4 hours as absolute values). This confirms that the formulation is effective and that the amount of dead cell debris is limited.
The proliferation capacity of the cells after 2 days of post-thawing re-culturing is greater than the positive control (+10.3%): which confirms their capacity for recovery/persistance after thawing.
The functionality of the cells is preserved, since their capacity for degranulation (=synthesis of cytokines which mediate their immune activity, in response to CD3/CD28 activation) is preserved (91.0% of the positive control in terms of recovery rate).
The percentage of CD25+ activated cells (part of the IL-2 receptor) is greater than the positive control (+94.6%) and also reflects the capacities for activation/proliferation of the cells.
Only the CD8/CD4 ratio is adversely affected (0.36 instead of 0.55) due to the decrease in the proportion of cytotoxic CD8+ lymphocytes, known to be more sensitive to freezing/thawing cycles. This moderate decrease is tolerated for the product.

### For the Formulation B:

The viability achieves 102.6% of the positive control and is maintained at 89.9% after 4 hours (i.e. 81% of viable cells upon thawing, and 71% after 4 hours).
The proliferation capacity of the cells over 2 days after thawing is greater than the positive control (112.4% in terms of recovery rate).
The functionality of the cells is preserved, since the capacity for degranulation (synthesis of cytokines in response to CD3/CD28 activation) is 87.0%.
The percentage of activated cells is greater than the positive control (+88.6%).
Only the CD8/CD4 ratio is adversely affected (0.36 instead of 0.55) due to an increased mortality of the cytotoxic CD8+ lymphocytes, which does not adversely affect the functionality of the cells.

### Conclusion:

This study demonstrates that the formulations A and B:
are better than the 10% DMSO+4% HSA negative control carried out in a Nalgene box, the freezing effectiveness and the stability of which are limited; and
are similar to the specifications of the non-frozen positive control (less than 15% difference relative to the fresh cells in 4% human albumin).

This demonstrates that the use of complex formulations with synergistic compounds made it possible to significantly improve the general characteristics of the cells upon thawing.

The benefit of these formulations lies in the following properties:
non-toxicity; that is to say ready to use after thawing, which makes it possible to overcome any pharmaceutical handling operations at the hospital,
long-lasting frozen stability of the cells (several months),
thawed stability of 4 hours, encompassing the preparation and handling time before injection of the patient,
compatibility with industrial storage constraints, and
preservation of the general and functional characteristics of the cells.

They therefore respond to common problems encountered in cell therapy.

1. A process for the cryopreservation of at least one sample of tumor-infiltrating lymphocytes, comprising the following steps:
i) in vitro culture of tumor-infiltrating lymphocytes from a sample of in-transit cutaneous nodules, lymph-node or metastasis originating from a patient suffering from a stage 3 or 4 melanoma, comprising the emergence of said tumor-infiltrating lymphocytes contained in the sample of in-transit cutaneous nodules, lymph-node or metastasis, then the stimulation of the tumor-infiltrating lymphocytes resulting from the emergence step, then finally the amplification of the stimulated tumor-infiltrating lymphocytes, in order to obtain at least one sample of tumor-infiltrating lymphocytes,
ii) mixing at least one sample obtained in step i) with a composition comprising, in a physiologically acceptable medium:
a) human serum albumin,
b) at least one saccharide, and
c) at least two ingredients selected from DMSO, L-cysteine, coenzyme Q10 and C3-C5 alkanediols,
iii) freezing the mixture obtained in step ii).

2. The cryopreservation process as claimed in claim 1, wherein the saccharide is selected from monosaccharides, disaccharides and trisaccharides.

3. The cryopreservation process as claimed in claim 2, wherein the saccharide is a disaccharide selected from trehalose and sucrose.

4. The cryopreservation process as claimed in claim 1, wherein the C3-C5 alkanediol is selected from propane-1,2-diol, pentane-1,5-diol and butane-2,3-diol.

5. The cryopreservation process as claimed in claim 1, wherein the human serum albumin is present in an amount of between 2 and 10% by weight relative to the total weight of composition.

6. The cryopreservation process as claimed in claim 1, wherein the saccharide is present at a concentration of between 0.05 M and 1 M.

7. The cryopreservation process as claimed claim 1, wherein the composition used in step ii) comprises:
a) human serum albumin,
b) trehalose, and
c) DMSO and L-cysteine.

8. The cryopreservation process as claimed in claim 1, wherein the composition used in step ii) comprises:
a) human serum albumin,
b) trehalose, and
c) coenzyme Q10 and propane-1,2-diol.

9. The cryopreservation process as claimed in claim 1, wherein the freezing iii) is carried out down to a temperature of between −100° C. and −180° C.

10. The cryopreservation process as claimed claim 1, wherein the freezing iii) is carried out by placing the mixture obtained in ii) in a container submerged in a mixture of isopropyl alcohol at +4° C., everything being brought to a temperature of between −70° C. and −100° C.

11. The cryopreservation process as claimed in claim 1, wherein the freezing iii) is carried out by means of a programmed freezer.

12. A method for the cryopreservation of at least one sample of tumor-infiltrating lymphocytes comprising the step of mixing a composition comprising, in a physiologically acceptable medium:
a) human serum albumin,
b) at least one saccharide, and
c) at least two ingredients selected from DMSO, L-cysteine, coenzyme Q10 and C3-C5 alkanediols,
said sample being obtained by in vitro culture of tumor-infiltrating lymphocytes from a sample of in-transit cutaneous nodules, lymph-node or metastasis originating from a patient suffering from a stage 3 or 4 melanoma, said culture comprising the emergence of said tumor-infiltrating lymphocytes contained in the sample of in-transit cutaneous nodules, lymph-node or metastasis, then the stimulation of the tumor-infiltrating lymphocytes resulting from the emergence step, then finally the amplification of the stimulated tumor-infiltrating lymphocytes.

13. The method as claimed in claim 12, wherein the tumor-infiltrating lymphocytes resulting from the emergence step are stimulated in the presence at least of non-proliferating irradiated allogeneic feeder cells.

14. The method as claimed in claim 12, wherein the tumor-infiltrating lymphocytes resulting from the emergence step are stimulated by coculture with a mixture formed of mononuclear cells originating from healthy human donor blood and of B cells transformed by the Epstein-Barr virus, all non-proliferating.

15. The cryopreservation process as claimed in claim 5, wherein the human serum albumin is present in an amount of between 2.5 and 6% by weight relative to the total weight of composition.

16. The cryopreservation process as claimed in claim 6, wherein the saccharide is present at a concentration of between 0.07 M and 0.5 M.

17. The cryopreservation process as claimed in claim 7, wherein the composition used in step ii) comprises:
a) the human serum albumin in an amount of between 2.5 and 6% by weight,
b) the trehalose at a concentration of between 0.07 M and 0.5 M, and
c) the DMSO and the L-cysteine respectively in an amount of between 2 and 15% by weight and at a concentration of between 0.5 mM and 2 mM.

18. The cryopreservation process as claimed in claim 8, wherein the composition used in step ii) comprises:
a) the human serum albumin in an amount of between 2.5 and 6% by weight,
b) the trehalose at a concentration of between 0.07 M and 0.5 M, and
c) the coenzyme Q10 and the propane-1,2-diol respectively in an amount of between 0.007 and 0.1% by weight and in an amount of between 3 and 10% by volume.

19. The cryopreservation process as claimed in claim 9, wherein the freezing iii) is carried out down to a temperature of between −140° C. and −160° C.
